(19) United States
(12) Patent Application Publication
(10) Pub. No.: US 2017/0304586 A1
(43) Pub. Date: Oct. 26, 2017

(54) METHOD FOR REDUCING TENSION IN MUSCLES

(71) Applicant: Joseph Oberholzer, Dallastown, PA (US)

(72) Inventor: Joseph Oberholzer, Dallastown, PA (US)

(21) Appl. No.: 15/137,324

(22) Filed: Apr. 25, 2016

(51) Int. Cl. A61M 21/02; A61M 21/00

(52) U.S. Cl. CPC A61M 21/02; A61M 21/00; A61H 2205/022; A61H 2205/065

(57) ABSTRACT

A method for relaxing muscles and reducing tension in a human body based on neurovascular diversion (NVD) which intends to reverse the flows of the neurological and vascular systems to effect changes in the musculoskeletal system. Neurological diversion occurs by interrupting the central nervous system's (CNS) autonomic sympathetic neurological flow to muscles of the axial spine and extremities. Vascular diversion occurs as the participant performs contractions using a most minimal force level in axial or peripheral muscle groups, which are neurologically linked to those in the hands and face to promote the shunting of blood to said muscle groups. The method is termed as low motion physio (LMP) which enables the participant to use inherent physiological processes to relax muscles and remove muscular tension in the human body.

### BACKGROUND OF THE INVENTION

### 1. Field of the Invention

The present disclosure generally relates to exercise techniques, which promote relaxation of muscular tension and more specifically to muscles in the human body by utilizing inherent physiological properties to promote blood flow to targeted muscle groups via a process called neurovascular diversion (NVD).

### 2. Description of Related Art

Skeletal muscles in the human body assist in movement and provide support for the axial skeleton and organs in order to perform a variety of motor activities. Skeletal muscles must have a normal resting length tension relationship for performing the aforementioned functions. When muscles are in a guarded state or spasm, blood flow is restricted due to extravascular compression by the inherent muscle fibers further inducing this state. Additionally, articular joints may become restricted causing a host of orthopedic dysfunction such as degenerative joint disease and repetitive use injuries to name a few.

A multitude of techniques exists to relieve muscular stiffness and spasms, to foster improved postural alignment, joint range of motion, muscle hydration, bone density, and overall musculoskeletal health. Examples might include stretching, strengthening, autogenic relaxation, visual imagery, deep breathing, hypnosis, massage, meditation, tai chi, yoga and biofeedback to highlight a few. A majority of these techniques require practice over an extended period in order to become proficient and, in many cases may require a skilled practitioner to effectively render these modalities.

In some cases, conventional techniques, which are designed to foster muscle relaxation, require the participant to assume various static and dynamic postures, which may be contraindicated or difficult for all participants to assume. Examples include those contending with acute pain, arthritis, sports related injuries, convalescents and pregnant women attempting stretching or yoga postures. Additionally, these methods may require participants to generate contractile forces, which also may be difficult or contraindicated for a variety of reasons. Furthermore, some of these methods may also require devices to be effective, which may incur expense and or cause injury.

Therefore, there is a need in the art for new and improved methods to decrease muscular tension. A method which almost any participant, irrespective of age or physical condition (barring immaturity, mental and cognitive deficits) would be able to perform with virtually no risk of injury and without the need of a device or assistance from another individual(s) or licensed professional(s).

### SUMMARY OF THE INVENTION

The present invention relates to a method which decreases muscular tension by promoting blood flow to targeted muscle groups through self-manipulation of the neurological and vascular systems. This is accomplished via a two part process known as Neurovascular Diversion (NVD).

First, neurological diversion (ND) occurs by interrupting the central nervous system's (CNS) autonomic sympathetic neurological flow to the axial and extremity musculature (heretofore referred to as the “body”). This is accomplished by simultaneously contracting muscles in more neurologically dense structures, namely the hands and face, thereby drawing neurological flow from less dense, neurologically linked muscles in the body (an example of neurological linkage is flexion in the hands, neck and face corresponds neurologically to flexion in the back, arms and legs similar to the state of flexion a baby is postured in when born). Therefore, by flexing the fingers and simultaneously lowering the eyebrows ND occurs causing an apparent relaxation of the flexors in the body preparing the muscles to accept an increase in blood flow via vascular diversion.

Second, vascular diversion occurs as the participant performs contractions at the most minimal of force level in targeted muscle group(s) in the body which are neurologically linked to the particular plane of motion in the hand and face that were performed during ND. In an embodiment, the targeted muscle group comprises specific muscle groups in the body including all planes of the bilateral upper and lower extremities, pelvis, lower, mid, upper back and neck.

The purpose of the minimal muscle contractions (subsequently referred to as a “pulse”) is so ensure that the muscle fibers do not compress the inherent vasculature and thus deprive themselves of blood flow. Following the completion of NVD, capillary perfusion is reestablished to deeper layers of muscle tissue which causes a decrease in muscular tension as evidenced by an apparent softening of the muscle tissue and decreased tenderness to palpation. NVD promotes realignment of bony segments and normalization of joint movement via normalization of the proper length tension relationship in the musculature of the body.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF SEVERAL VIEWS OF DRAWINGS

FIG. 1A illustrates a schematic representation of neurological diversion of the extensor muscles (back) of the body via contractions of the hand and face extensors. FIG. 1B through FIG. 1G illustrates a schematic representation for relaxation of the extensor muscles of the body via vascular diversion.

FIG. 2A illustrates a schematic representation of neurological diversion of the flexor muscles (front) of the body via contractions of the hand and face flexors.

FIG. 2B through FIG. 2G illustrates a schematic representation for relaxation of the flexor muscles of the body via vascular diversion.

FIG. 3A illustrates a schematic representation of neurological diversion of muscles in the abductor (side) muscles of the body via contractions of the hand and face abductors.

FIG. 3B through FIG. 3E illustrates a schematic representation of vascular diversion of muscles in the abductor (side) muscles of the body via contractions of the hand and face abductors.

FIG. 4A illustrates a schematic representation of neurological diversion of muscles in the adductor (medial leg) muscles via contractions of the hand and face adductors.

FIG. 4B through FIG. 4E illustrates a schematic representation for relaxation of muscles in the adductor (medial leg) via vascular diversion.

Please refer to FIG. 1A which illustrates a schematic representation of neurological diversion of the left lumbar and thoracic rotator muscles of the body via contractions of the hand and face extensors.

FIG. 5A and FIG. 5B illustrates a schematic representation for relaxation of muscles of the left lumbar and thoracic rotator muscles via vascular diversion.

Please refer to FIG. 1A which illustrates a schematic representation of neurological diversion of the left middle back extensor muscles of the body via contractions of the hand and face extensors.

FIG. 6A through 6C illustrates a schematic representation for relaxation of the left middle back extensor muscles via vascular diversion.

Please refer to FIG. 1A which illustrates a schematic representation of neurological diversion of the left upper back extensor muscles of the body via contractions of the hand and face extensors.

FIG. 7A through FIG. 7C illustrates a schematic representation for relaxation of the left upper back extensor muscles via vascular diversion.

Please refer to FIG. 1A which illustrates a schematic representation of neurological diversion of the neck extensor muscles of the body via contractions of the hand and face extensors.

FIG. 8A through FIG. 8C illustrates a schematic representation for relaxation of the neck extensor muscles of the posterior (back) of the neck via vascular diversion.

Please refer to FIG. 1A which illustrates a schematic representation of neurological diversion of extensor muscles in the posterior of the arm via contractions of the hand and face extensors.

FIG. 9A and FIG. 9B illustrates a schematic representation for relaxation of the extensor muscles in the posterior of the left arm via vascular diversion.

Please refer to FIG. 2A which illustrates a schematic representation of neurological diversion of the flexor muscles in the anterior of the arm via contractions of the hand and face flexors. FIG. 10A and FIG. 10B illustrates a schematic representation for relaxation of the flexor muscles in the anterior of the arm via vascular diversion.

Please refer to FIG. 3A which illustrates a schematic representation of neurological diversion of abductor muscles in the lateral aspect of the left arm via contractions of the hand and face abductors.

FIG. 11A and FIG. 11B illustrates a schematic representation for relaxation of abductor muscles in the lateral aspect of the left arm via vascular diversion.

Please refer to FIG. 4A which illustrates a schematic representation of neurological diversion of adductor muscles in the medial aspect of the left arm via contractions of the hand and face adductors.

FIG. 12A and FIG. 12B illustrates a schematic representation for relaxation of adductor muscles in the medial aspect of the left arm via vascular diversion.

FIG. 13A through FIG. 13C illustrates a schematic representation for stretching of the left axial extensors.

FIG. 14A through FIG. 14C illustrates a schematic representation to re-coordinate muscles in the anterior flexors of left leg via muscle sequencing.

FIG. 15A illustrates a schematic representation for the mobilization of the left ankle (Talus) bone. FIG. 15B illustrates a schematic representation for the mobilization of the right ankle (Talus) bone.

### DETAILED DESCRIPTION OF DIFFERENT EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is therefore indicated by the appended claims rather than by the foregoing description. All changes that come within the meaning and range of equivalency of claims are to be embraced within their scope.

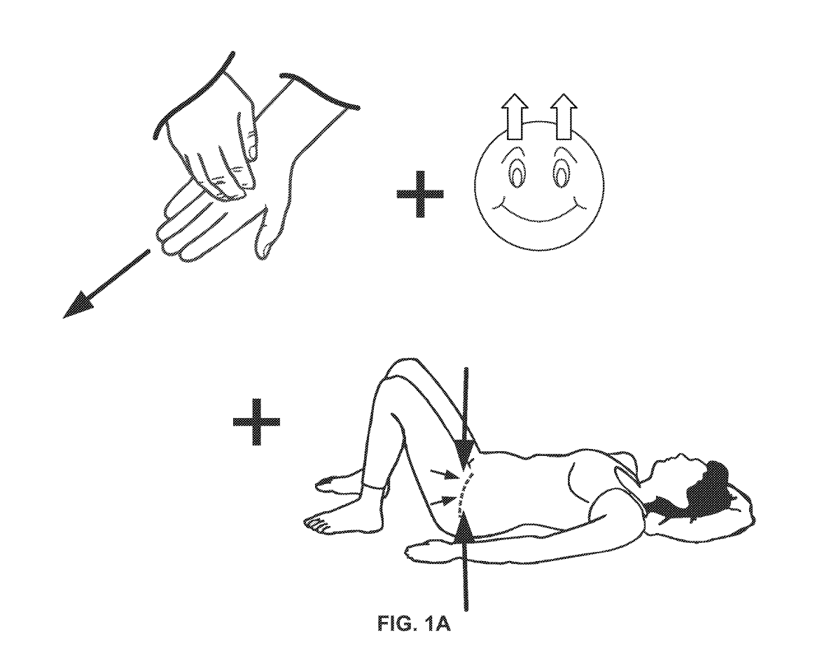

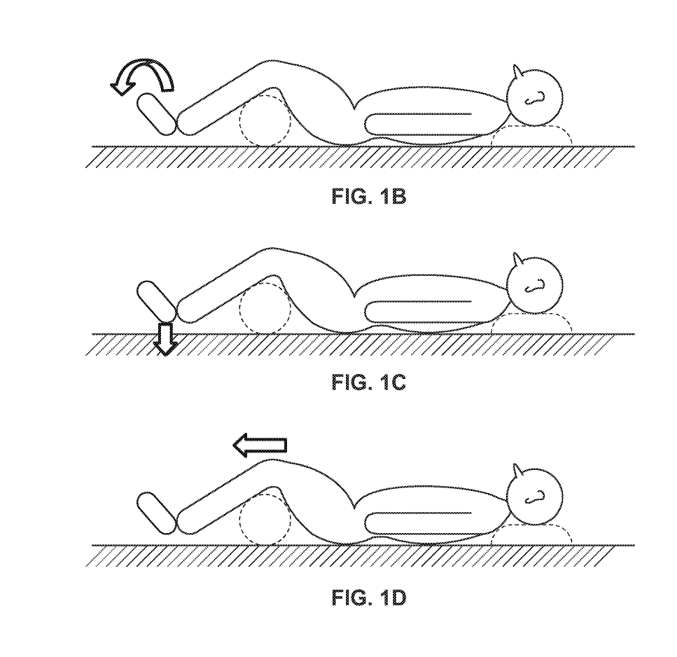

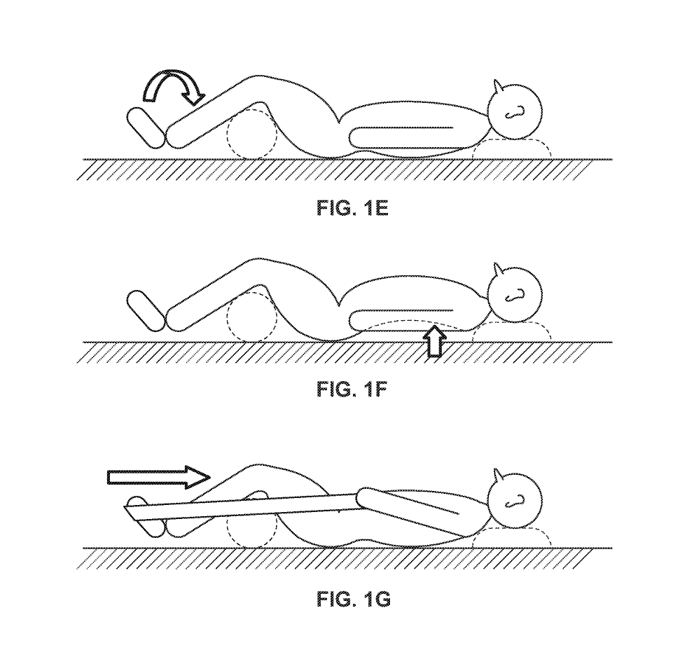

FIG. 1A illustrates a schematic representation for relaxation of muscles in the posterior (back) aspect of the left leg via neurological diversion. The relaxation can be achieved by simultaneously uncurling (extending) LEFT fingers, contracting the muscles of the pelvic floor and smiling with tongue touching on roof of mouth and raising eyebrows in a pulse rhythm as shown in the FIG. 1A. The above combination of activities can be performed with 20 repetitions.

FIG. 1B through FIG. 1G illustrates a schematic representation for relaxation of muscles in the posterior (back) aspect of the left leg via vascular diversion. FIG. 1B illustrates tapping (plantar flex) left foot down towards floor when lysing on a flat surface. FIG. 1C illustrates bending (flex knee) by pushing left heel into bed. FIG. 1D illustrates extending left hip, left leg towards foot of the bed. FIG. 1E illustrates rotating hip laterally (log rolling) with left leg outwards. FIG. 1F illustrates arching of the back (axial extension). FIG. 1G illustrates stretching using a strap to pull (dorsiflex) left foot towards head in a pain free range of motion for 1 repetition of 10 second hold time.

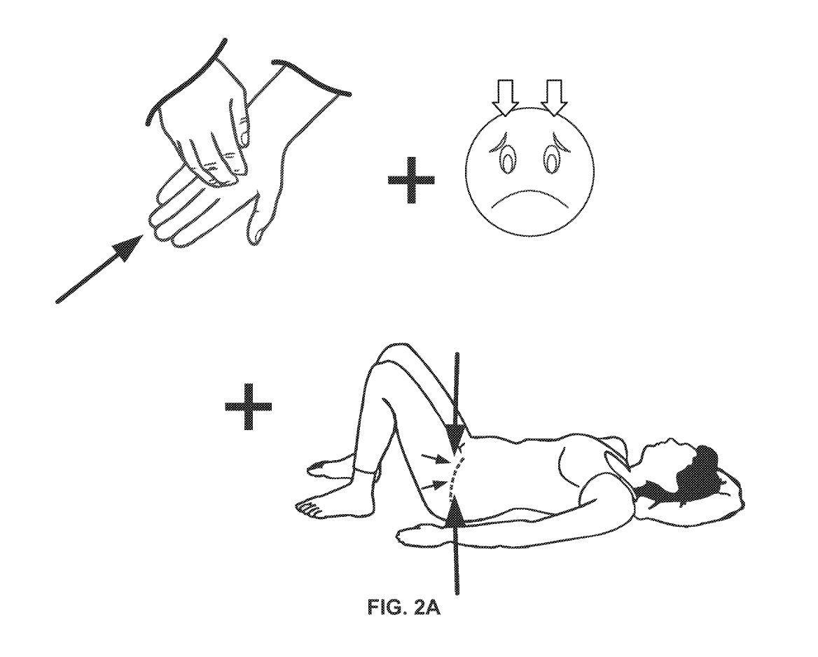

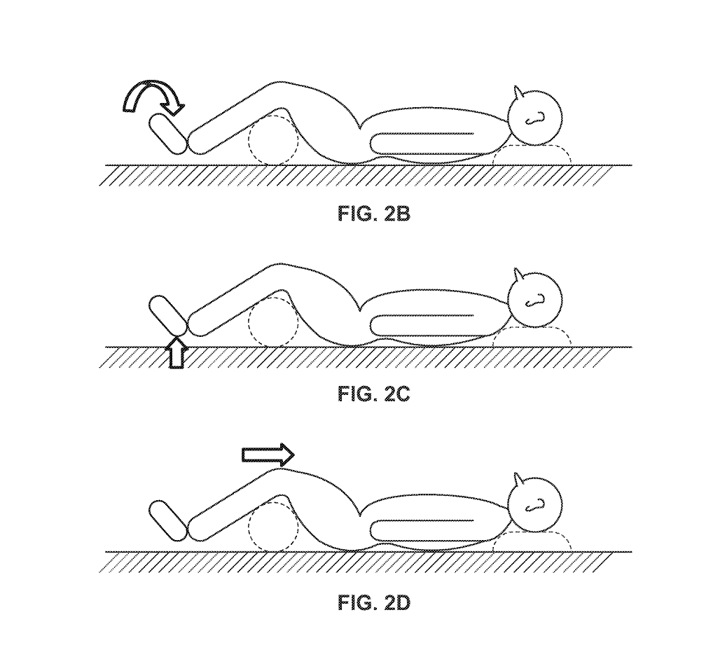

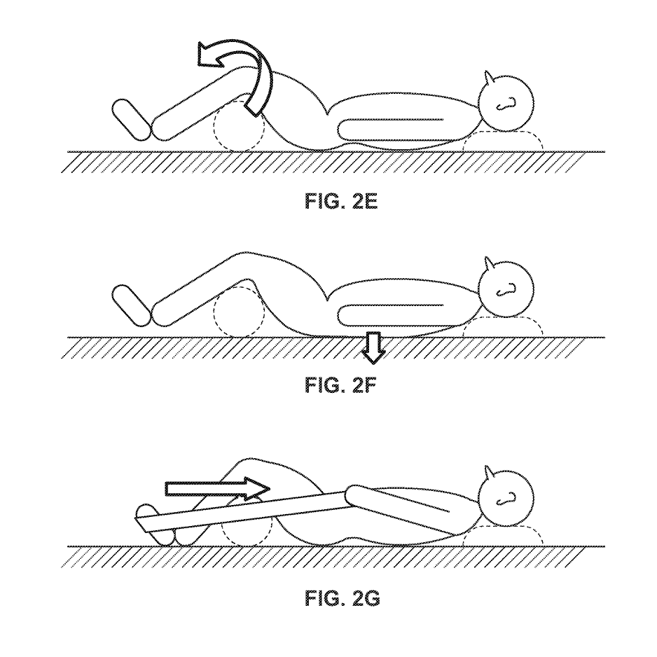

FIG. 2A illustrates a schematic representation for relaxation of muscles in the anterior (front) aspect of the left leg via neurological diversion. The relaxation can be achieved by simultaneously curling (flexing) left fingers, contracting the muscles of the pelvic floor, frowning with tongue on floor of mouth and lowering eyebrows in a pulse rhythm for 20 repetitions.

FIG. 2B through FIG. 2G illustrates a schematic representation for relaxation of muscles in the anterior (front) aspect of the left leg via vascular diversion. FIG. 2B illustrates moving (dorsiflex) left foot towards head. FIG. 2C illustrates beginning to lift (extending knee) left heel but not off surface. FIG. 2D illustrates beginning to move (flexing hip) left knee towards head without moving the leg. FIG. 2E illustrates log rolling (rotating hip medially) with left leg inward. FIG. 2F illustrates flattening back (axial flexion) of the body. FIG. 2G illustrates stretching using a sheet, pulling (dorsiflex) the left foot towards head and bending (flexing) knee with strap in a pain free range of motion for 1 repetition of 10 second hold time.

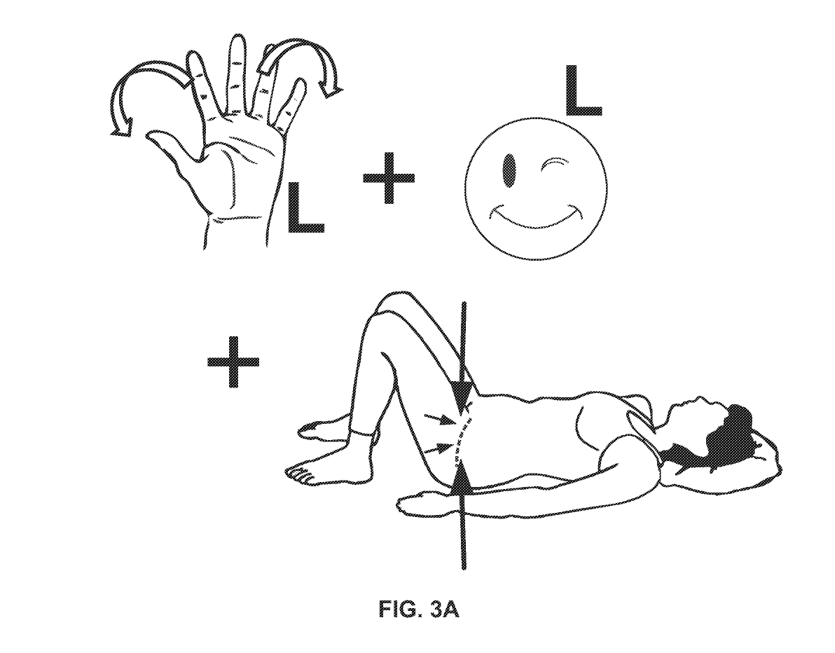

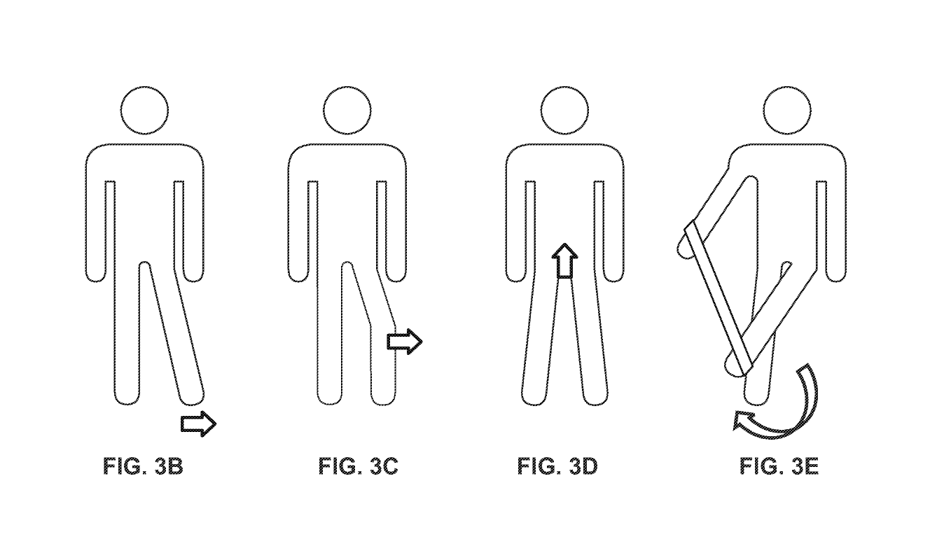

FIG. 3A illustrates a schematic representation for relaxation of muscles in the lateral (side) aspect of the left leg via neurological diversion. The relaxation can be achieved by simultaneously opening (abduct) left fingers apart, contracting the muscles of the pelvic floor and left cheek with tongue resting against the inside of the left molars of mouth in a pulse rhythm, for 20 repetitions.

FIG. 3B through FIG. 3E illustrates a schematic representation for relaxation of muscles in the lateral (side) aspect of the left leg via vascular diversion. FIG. 3B illustrates moving (abduct ankle) left foot to the left. FIG. 3C illustrates moving (abduct hip) left knee to the left. FIG. 3D illustrates moving (elevating) left hip upward and FIG. 3E illustrates stretching using a strap to move (adduct hip) left leg to the right in a pain free range of motion for 1 repetition of 10 second hold time.

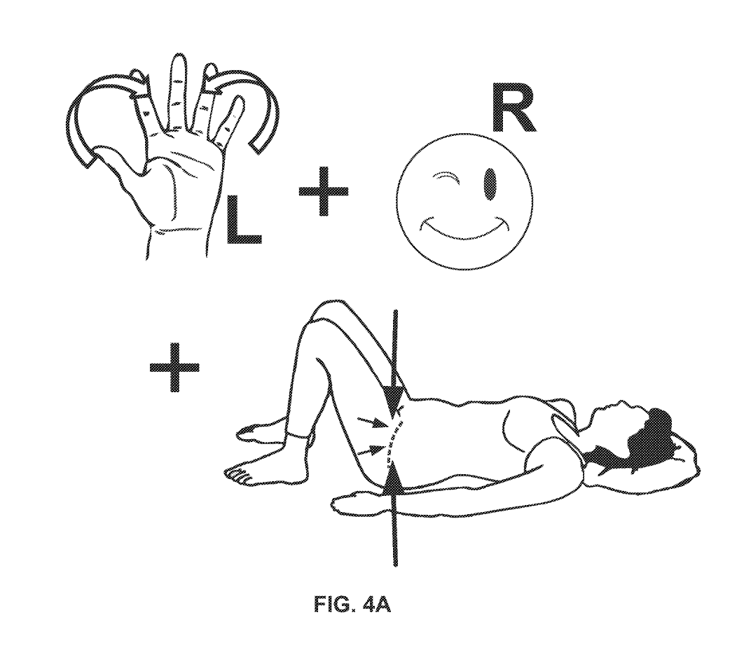

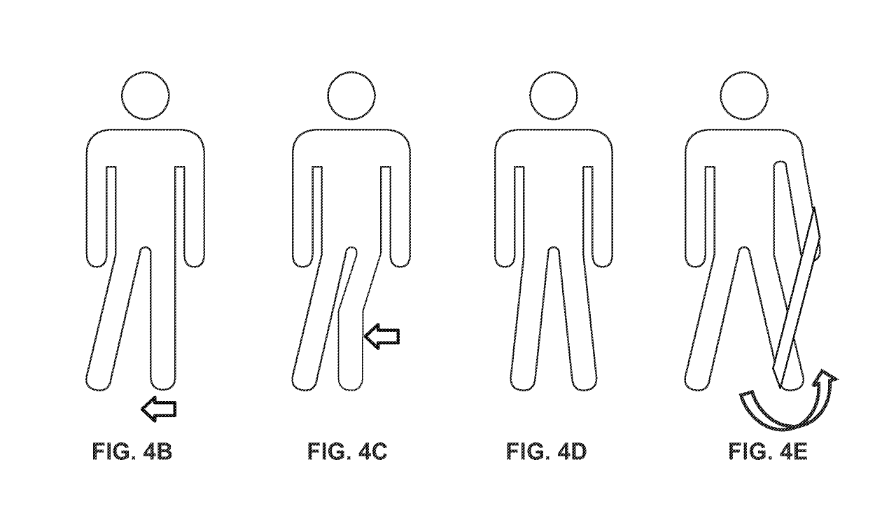

FIG. 4A illustrates a schematic representation for relaxation of muscles in the medial (inside) aspect of the left leg via neurological diversion. The above relaxation can be achieved by simultaneously closing (adduct) left fingers apart, contracting the muscles of the pelvic floor and right cheek with tongue resting against the inside of the right molars of the mouth in a pulse rhythm for 20 repetitions.

FIG. 4B through FIG. 4E illustrates a schematic representation for relaxation of muscles in the medial (inside) aspect of the left leg via vascular diversion. FIG. 4B illustrates moving (adduct ankle) left foot to the right. FIG. 4C illustrates moving (adduct hip) left knee to the right. FIG. 4D illustrates contracting muscles of the pelvic floor. FIG. 4E illustrates stretching using a strap to move (abduct hip) left leg to the left in a pain free range of motion for 1 repetition of 10 second hold time.

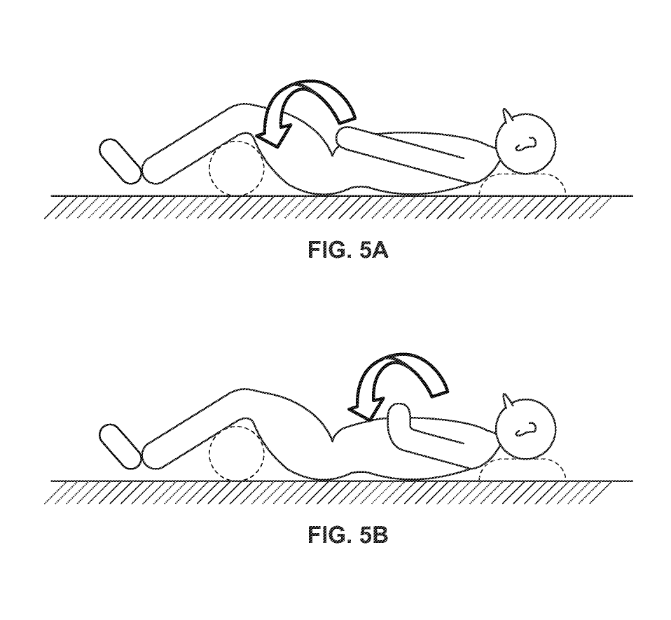

FIG. 5A and FIG. 5B illustrates a schematic representation for relaxation of muscles of the left lumbar and thoracic rotator muscles via vascular diversion. FIG. 5A illustrates arms folded on lower abdomen, and moving (rotating) pelvis to the LEFT for 20 times. FIG. 5B illustrates a position with arms folded on chest, and moving (rotating) upper body to the LEFT 20 times.

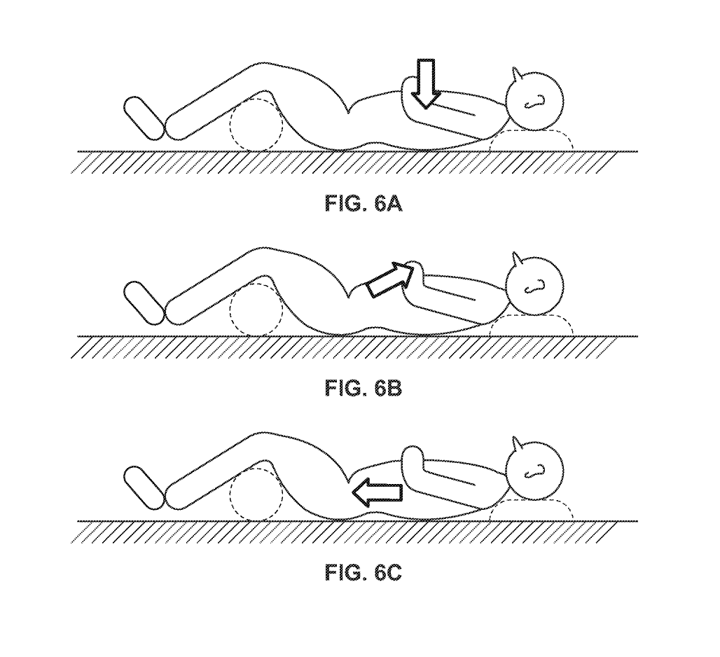

FIG. 6A through FIG. 6C illustrates a schematic representation for relaxation of the left middle back muscles (latissimus muscle) via vascular diversion. FIG. 6A illustrates moving left arm down towards floor (extending shoulder). FIG. 6B illustrates moving LEFT arm to the right (adduct shoulder). FIG. 6C illustrates moving left arm toward foot (depressing shoulder).

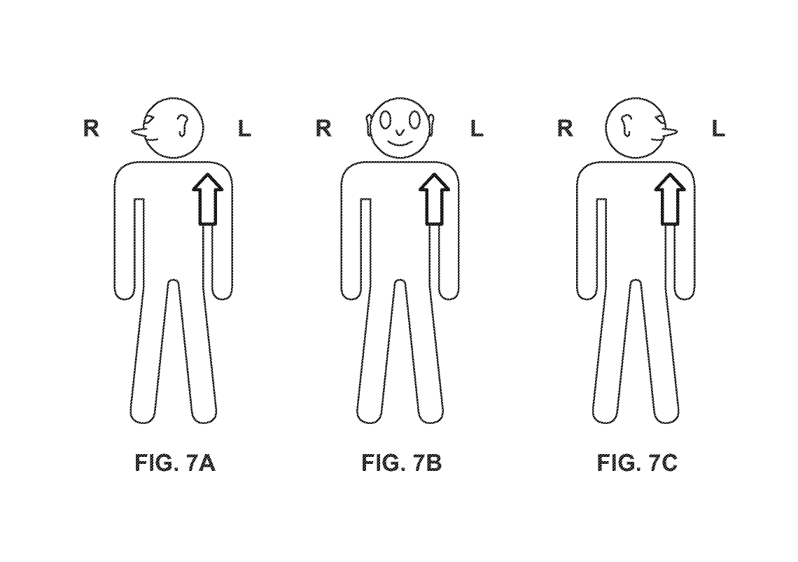

FIG. 7A through FIG. 7C illustrates a schematic representation for relaxation of the left upper back (trapezius) muscles via vascular diversion. FIG. 7A illustrates rotating of the head with right shrug and elevating the left shoulder. FIG. 7B illustrates head being centered in midline and shrugging (elevate) left shoulder. FIG. 7C illustrates head being rotated to left with shrugging or elevating the left shoulder.

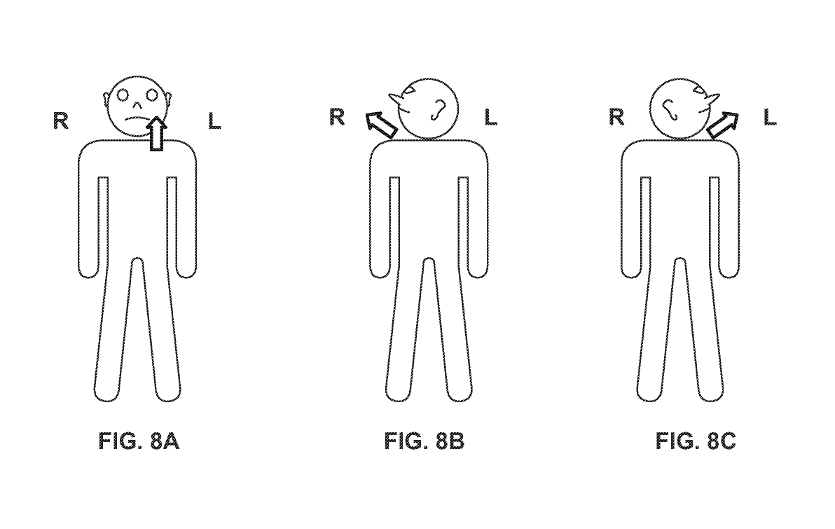

FIG. 8A through FIG. 8C illustrates a schematic representation for relaxation of the neck extensor muscles of the posterior (back) of the neck via vascular diversion. FIG. 8A illustrates head centered in midline and nodding (extending) the head upward. FIG. 8B illustrates head rotated to right and nodding (extending) head upward. FIG. 8C illustrates head rotated to left and nodding (extending) head upwards.

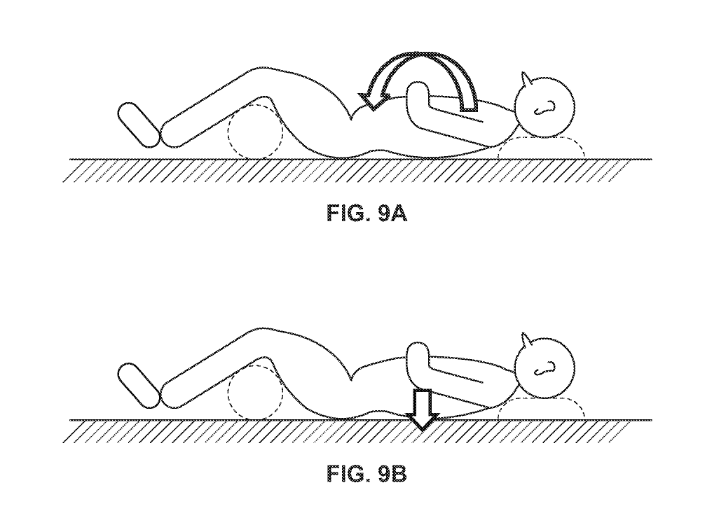

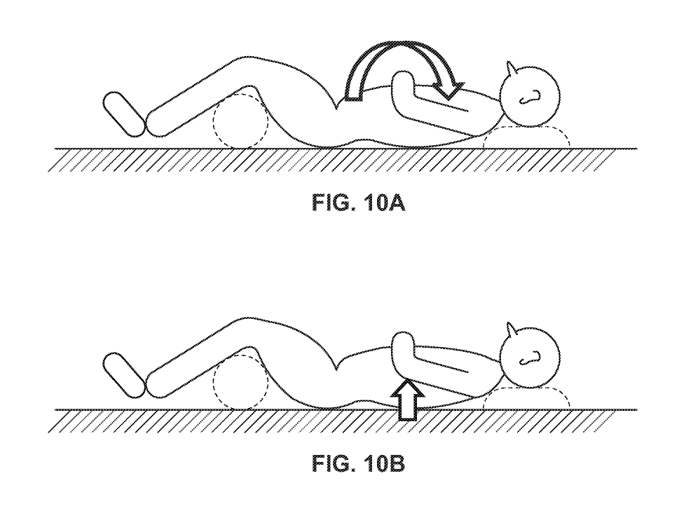

FIG. 9A and FIG. 9B illustrates a schematic representation for relaxation of the muscles in the back of the left arm via vascular diversion. FIG. 9A illustrates straightening (extending elbow) left lower arm. FIG. 9B illustrates moving left arm towards floor (shoulder extension).

FIG. 10A and FIG. 10B illustrates a schematic representation for relaxation of the muscles in the front of the arm via vascular diversion FIG. 10A illustrates bending (flexing elbow) left lower arm. FIG. 10B illustrates moving left lower arm towards ceiling.

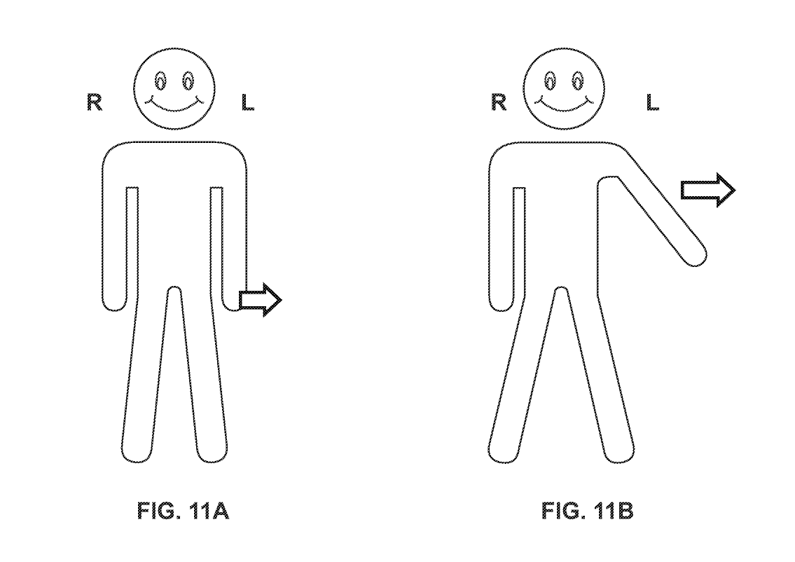

FIG. 11A and FIG. 11B illustrates a schematic representation for relaxation of muscles in the lateral (side) aspect of the left arm via vascular diversion. FIG. 11A illustrates moving (abduct wrist) left hand to the left. FIG. 11B illustrates moving (abduct shoulder) left arm to the left.

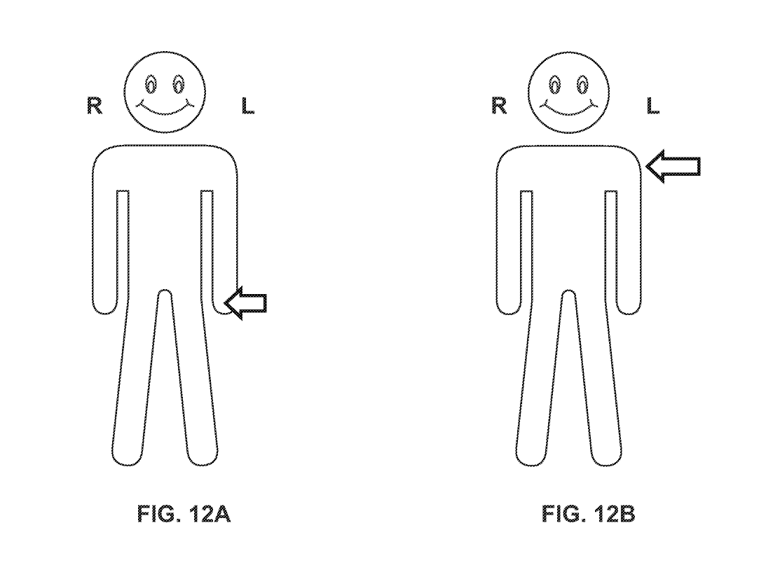

FIG. 12A and FIG. 12B illustrates a schematic representation for relaxation of muscles in the medial (inside) aspect of the left arm via vascular diversion. FIG. 12A illustrates moving (adduct wrist) left hand to the right. FIG. 12B illustrates moving (adduct shoulder) left arm to the right.

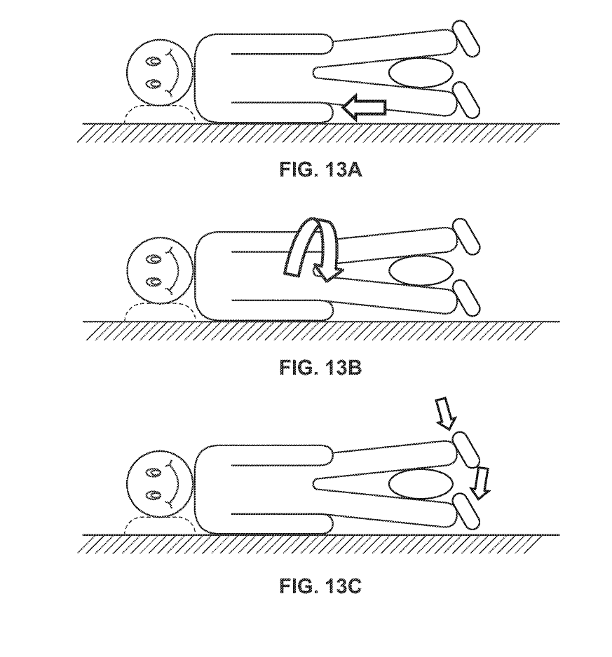

FIG. 13A through FIG. 13C illustrates a schematic representation for stretching of the left axial extensors. FIG. 13A illustrates side lying on right by moving (flex bilateral hips) both legs toward head (superiorly) by 1 inch. FIG. 13B illustrates rotating pelvis forward (anteriorly) 1 inch and FIG. 13C illustrates moving (Left hip adduction and right hip abduction) both ankles toward floor with 1 pound of pressure and holding for 10 seconds.

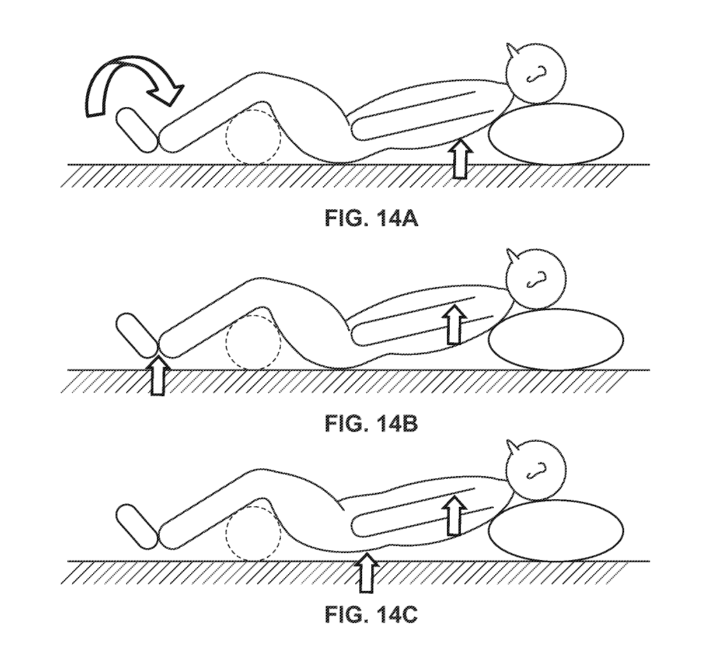

FIG. 14A through FIG. 14C illustrates a schematic representation to re-coordinate muscles in the anterior (front) left leg via muscle sequencing. FIG. 14A illustrates moving (dorsiflex) left foot towards head while holding an isometric contraction of the right shoulder into flexion and holding the contraction for 3 seconds. FIG. 14B illustrates straightening left knee (extending knee) while continuing to hold an isometric contraction of the right shoulder into flexion and holding contractions simultaneously with the position as shown in FIG. 14A. In addition, FIG. 14C illustrates contracting left buttock muscle and holding contraction simultaneously with the movements as shown in FIG. 14A and FIG. 14B.

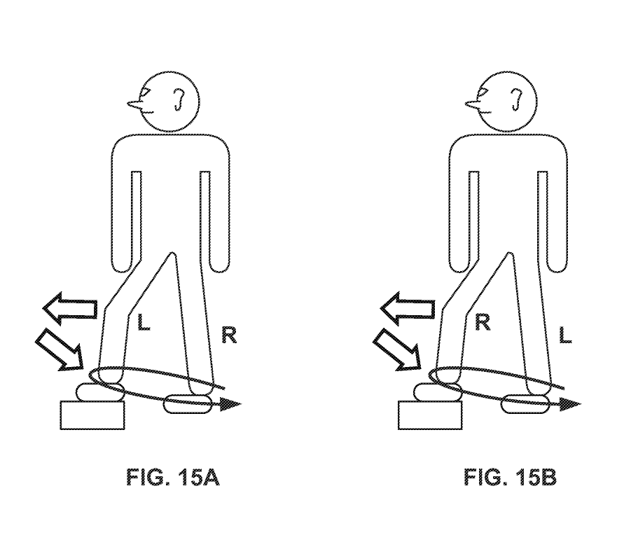

FIG. 15A illustrates a schematic representation for the mobilization of the left ankle (Talus) bone. FIG. 15B illustrates a schematic representation for the mobilization of the right ankle (Talus) bone.

The prior art techniques of muscle relaxation and tension reduction, require a minimum of focus and coordination and would provide best results if performed on a daily basis for an extended period but can be stopped or paused at any time if the user feels any kind of discomfort or pain. Other ways of muscle relaxation include administration of medications, muscle stretching, muscle strengthening and joint mobilization. On the other hand, the techniques of the present invention utilize minute muscle contractions at the lowest force level possible, preferably done while lying down, which minimizes chances of incurring an injury. Additionally, the present invention can be performed in any position by individuals of almost any age and activity level barring immaturity or cognitive deficits. Other advantages include that special devices or licensed professionals/trainers are not required to assist the participant in the performance of the above techniques.

In an embodiment, the techniques of the present invention have therapeutic effects on an individual by reducing stress and tension, improving user's flexibility, improving muscle tone, overall wellbeing, improving sleeping patterns and increasing the effectiveness of overall health.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation in the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A method for relaxing muscles and reducing tension in the human body, the method comprising the steps of:
i) contracting muscles in hands and face, which are linked neurologically in the same contractile pattern as muscles in the axial skeleton and extremities thereby shifting the neurological focus from the axial skeleton and the extremities to the hands and face.
ii) performing low force level contractions of muscle groups in the axial spine and or extremities so that blood may be shunted and perfuse into the deeper levels of the muscle tissue unhindered by extravascular compression of the muscle fibers, inducing relaxation of those same muscle groups.

2. The method of claim 1, wherein the target muscle group comprises muscles present in left leg and right leg.

3. The method of claim 1, wherein the target muscle group comprises muscles present in lower, upper back and neck regions of the human body.

4. The method of claim 1, wherein the target muscle group comprises muscles present in outside, inside, front side and back side of left leg and right leg.

5. The method of claim 1, wherein the plurality of muscles are selected from a group consisting of head, tongue, lips, fingers, toes and pelvic musculature.

6. The method of claim 1, wherein the plurality of muscles are selected from a group consisting ankles, knees, hip, upper body and neck musculature.

7. The method of claim 1, further allows bone realignment and restoration of joint movement.
